(19) United States
(12) Patent Application Publication
(10) Pub. No.: US 2010/0168856 A1
(43) Pub. Date: Jul. 1, 2010

(54) Multiple piece tissue void filler

(75) Inventors: Marc Long, Denville, NJ (US); Twana Davisson, Montclair, NJ (US)

Correspondence Address: LERNER, DAVID, LITTENBERG,;KRUMHOLZ & MENTLIK, 600 SOUTH AVENUE WEST, WESTFIELD, NJ, 07090, US

(73) Assignee: Howmedica Osteonics Corp., Mahwah, NJ (US)

(21) Appl. No.: 12/319,128

(22) Filed: Dec. 31, 2008

(51) Int. Cl. A61F 2/02

(52) U.S. Cl. 623/14.12

(57) ABSTRACT

A method for the repair of a cartilage defect in a patient in need thereof, including implanting an implant into the cartilage defect, wherein the implant may comprise at least a first material, wherein the first material may be porous and may be a scaffold that is expandable or compressible. The invention also includes an implant for the repair of a cartilage defect in a patient in need thereof, the implant may include at least a first material and a second material, wherein the first material may be porous and may be a scaffold that is expandable or compressible, and wherein the first material may surround the second material.

### FIELD OF THE INVENTION

The present invention relates to the field of medical technology and is generally directed to the treatment of cartilage or cartilage and bone defects through the use of grafts, scaffolds, graft and scaffold combinations, and the like.

### BACKGROUND OF THE INVENTION

Cartilage is an avascular connective tissue made up of collagen and/or elastin fibers, and chondrocytes, all of which are embedded in a matrix. There are three main types of cartilage: elastic, fibrocartilage, and hyaline. Elastic cartilage is found in the outer ear and the epiglottis. Fibrocartilage is found between the bones of the spinal column, hips and pelvis. Hyaline cartilage can be found on the ends of bones which form joints, on the ends of the ribs, on the end of the nose, on the stiff rings around the windpipe, and supporting the larynx. Articular cartilage is a specialized type of hyaline cartilage which covers the surface of joints and provides a durable low friction surface that distributes mechanical forces and protects the joint's underlying bone.

Different types of collagen can be found in varying amounts in the collagen matrix, depending on the type of tissue. For example, hyaline cartilage, which is found predominantly in articulating joints, is composed mostly of type II collagen with small amounts of types V, VI, IX, X, and XI collagen also present. On the other hand, fibrocartilage, which can also be found in joints, is primarily composed of type I collagen. Additionally, the fibrocartilaginous tissue that sometimes replaces damaged articular cartilage is composed of type I collagen.

Loss of or damage to cartilage can lead to painful conditions such as osteoarthritis. Damage to cartilage can be caused by traumatic injury, disease and/or age. Since cartilage lacks nerves and blood vessels, it has very limited regenerative capabilities compared to other tissues. Consequently, the healing of damaged joint cartilage results in a fibrocartilaginous repair tissue that lacks the structure and biomechanical properties of normal cartilage. Over time, the repair tissue degrades and leaves damaged joint cartilage, which causes osteoarthritis and reduced movement in the joint.

There is a need for methods for repairing cartilage defects.

### BRIEF SUMMARY OF THE INVENTION

The present invention includes an implant that can be used to repair cartilage and methods of producing the implant. The invention also includes a method of treating cartilage defects using the implant.

The present invention includes, in one embodiment, a method for the repair of a cartilage defect in a patient in need thereof, including implanting an implant into the cartilage defect, wherein the implant comprises a first material, wherein the first material may be porous and may be a scaffold that is expandable and/or compressible. The first material may be, for example, demineralized bone matrix.

The invention also includes an implant for the repair of a cartilage defect in a patient in need thereof, comprising a first porous material and a second material, wherein the first material may be expandable or compressible, and wherein the first material may partially or substantially surround the second material. The second material may be, for example, porous and expandable and/or compressible.

Furthermore, the implant comprises a first material that is porous and compressible and/or expandable. The implant can be used as both a scaffold for ex vivo tissue growth or as an implant used to repair tissue defects. The material used in the implant can be implanted alone or in combination with a second material, cells and/or biological factors at the time of surgery. Specifically, as to cartilage, the implant may be used for chondral, osteochondral, partial or full repair of cartilage defects.

### BRIEF DESCRIPTION OF THE DRAWINGS

FIG. 1 shows the compressive properties of the material of the invention.

FIG. 2. shows the expansion of the material of the invention in a cartilage defect.

FIG. 3. shows DBM graft filled defects (A and B) and autograft-filled defects (C) after 3 months.

FIG. 4. shows Safranin-O staining of cross sections of goat joints at 3 weeks post-implantation with DBM.

FIG. 5. shows various arrangements of the implant, where a second material is illustrated as a graft.

FIG. 6 shows one embodiment of the present invention in which the first material is rolled on itself to form a spiral, “centipede,” or “snail-shell” configuration.

FIGS. 7a-b. shows various embodiments of the present invention in which the first material substantially surrounds the second material, forming a cylindrical shape.

FIG. 8. shows other embodiments of the implant of the present invention including shapes such as conical, trapezoidal, tear-drop and T-shaped.

FIG. 9 shows one embodiment of a single defect site filled with multiple implants, wherein this example, the defect site is filled with three implants.

FIG. 10. shows further embodiments of the implant of the present invention including various tooth cap shapes.

FIG. 11 illustrates an empty osteochondral defect (a), an implant, in the form of a scaffold, within an applicator tool (b), and the implant within the defect (c).

FIG. 12 illustrates a scaffold implant made of DBM within a defect (a) and autograft filled defect (b) at 6 months.

FIG. 13 shows the integration score of both the scaffold filled defect and the autograft filled defect, based on histological analysis at 6 months.

FIG. 14 shows one embodiment of a macroscopic appearance of two examples of an autograft and an implant (labeled “scaffold”) at 3 months, 6 months, and 12 months in a trochlear groove defect.

FIG. 15 illustrates the macroscopic score for both the implant and the autograft, shown in FIG. 14, at 3 months, 6 months and 12 months.

FIG. 16 shows one embodiment of a macroscopic appearance of two examples of an autograft and an implant (labeled “scaffold”) at 3 months, 6 months, and 12 months in a condyle defect.

FIG. 17 illustrates the macroscopic score for both the implant and the autograft, shown in FIG. 16, at 3 months, 6 months and 12 months.

### DETAILED DESCRIPTION OF THE INVENTION

The present invention is directed to the repair of cartilage and may include, for example, a cartilage graft, scaffold, or combination of the two, and a method of repairing a cartilage defect using the cartilage graft, scaffold or combination of the two.

Although a number of different therapeutic methods are currently being used to treat cartilage defects, they have only been marginally successful. Some of the current treatments include lavage, arthroscopic debridement, and repair stimulation. However, these therapeutic methods either provide only temporary pain relief or have shown limited clinical efficacy.

Other treatment methods involve grafting the defect site with artificial materials, autografts, allografts, or xenografts. Examples of different grafts and grafting methods can be found in U.S. Pat. Nos. 5,944,755; 5,782,915; 6,858,042; 2003/0229400; and 2004/0230303, the disclosures of which are incorporated by reference herein. Grafts for cartilage repair include porous materials, such as PLA, collagen “sponges”, hyaluronic acid, metals (CoCr, Titanium), PVA, autograft, and allograft osteochondral plugs. None of these materials are both porous and expandable or compressible to a significant amount of their original size.

One particular grafting method, called mosaicplasty, has shown some clinical efficacy. Mosaicplasty involves removing small autologous osteochondral plugs from low weight bearing sites in a patient's joint. The osteochondral plugs are then grafted into a mosaic of holes drilled into the patient's articular cartilage defect site. Some patients who have undergone mosaicplasty have reported decreased pain and improved joint function. Marcacci, M. et al., Arthroscopy 21(4): 462-470 (2005).

Although all of the above methods have had some clinical success, each one of these therapeutic methods suffer from one or more of the following disadvantages: the risk of patient immune response or disease transmission; limited availability of osteochondral autograft sites; lack of implant adhesion to the defect site; implant deterioration; lack of long-term efficacy; donor site morbidity; patient discomfort; and the failure to restore normal joint function.

The Osteosponge™ (Bacterin International, Inc.; Belgrade, Mont.) has been developed for bone defects. It is a porous, compressible and expandable demineralized bone matrix (DBM), which has been shown to be useful as a scaffold for bone repair. The present inventors have shown that the Osteosponge™ can also be used for cartilage repair. The compressible and expandable DBM sponge is porous and can be compressed to 30% of its size prior to implantation. See the following U.S. Publications and Issued Patents for similar products: 2006/0085075; 2005/0090899; 2004/0115240; 2004/0197375; 20040062753; 20040166169; 20040078090; U.S. Pat. Nos. 7,056,337; 6,121,042; 6,319,712; 6,171,610; 5,882,929; and 6,124,273.

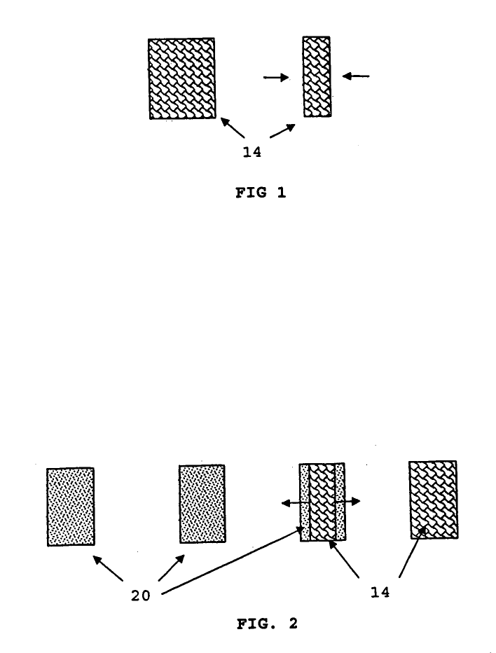

As shown in U.S. Application Publication No. 2008/0039954, the disclosure of which is hereby incorporated by reference herein, a graft comprising a material with sponge-like properties similar to Osteosponge™ is used in the repair of cartilage defects (chondral or osteochondral). The graft, which was similar to the graft 14 illustrated in FIGS. 1-4, allows, in this example, cartilage growth, resulting in restoration of function. The disclosure was also directed to a method for cartilage repair comprising implanting a graft into a cartilage defect in a patient, wherein the graft comprises a porous material which is also expandable and/or compressible. The material is porous, to allow in-growth of cells. The material is also compressible and/or expandable for better press-fit and chondro-integration.

In the present invention, an implant, such as a graft, scaffold, or combination of the two, comprising at least a material with sponge-like properties similar to Osteosponge™ is used in the repair of cartilage defects (chondral or osteochondral). The implant allows cartilage growth, resulting in restoration of function.

Thus, the present invention is directed to a method for cartilage repair comprising implanting an implant, into a cartilage defect site in a patient, wherein the implant, comprises a porous material which is also expandable and/or compressible. The material is porous, to allow in-growth of cells. The material is also compressible and/or expandable for better press-fit and chondro-integration.

In the case of a small tissue defect, for example, a defect that is up to and including about 1 cm², a single implant, such as illustrated in FIG. 1, may be sufficient to completely fill the defect. Thus, as illustrated in FIG. 1, graft 14, individually, may completely fill the tissue void 20.

However, in the case of tissue defects larger than about 1 cm², an implant including two or more pieces may be required to completely fill the defect.

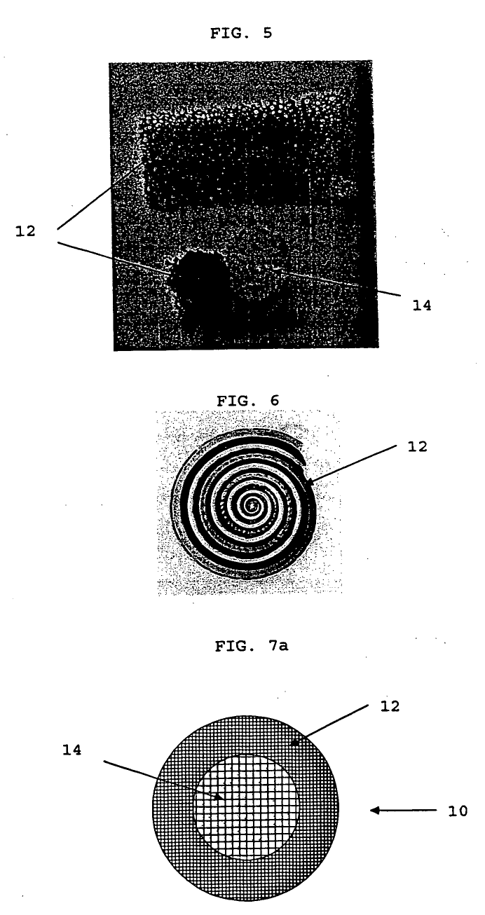

As illustrated in FIG. 5, in one embodiment of the present invention, a first material 12 may be a scaffold which may be expandable and/or compressible. The scaffold may be in various shapes, including, but not limited to, cylindrical, flat sheet, hexagonal, spherical, conical, X-shaped, T-shaped, tear-drop shaped, trapezoidal, or the like. Specifically, the scaffold may be in any shape required to fill a tissue defect or void completely. In FIG. 5, the first material 12 is illustrated to be a flat sheet and a cylindrical shape. The cylindrical shape of the first material 12 may be, for example, a flat sheet rolled on itself as in FIG. 6, or it may be formed directly into the shape of a cylinder. Likewise, the flat sheet shape may be folded on itself to form an oblong or oval shape. Shapes such as these may be used to fill larger defects. In the example of the flat sheet rolled on itself, the first material 12 forms a spiral, or “snail-shell” design which may provide additional expandability and compressibility and may be useful to fill larger defects.

In one embodiment, the scaffold is made of DBM, and may further specifically be similar to OsteoSponge™. The DBM is porous, compressible, and/or expandable, and is thus suitable for a tight press-fit application. The DBM may also be compressible and/or expandable in all dimensions, thus creating a more malleable material which can be used in a variety of locations and applications.

In larger tissue defects, such as those over 1 cm², the first material 12 may be combined with at least a second material 14. The first material 12 and second material 14 may be made from the same material or from different materials, and may be the same shape or different shapes. In one embodiment, second material 14 may be any type of graft, some examples of which were mentioned above, which may be found currently in the art.

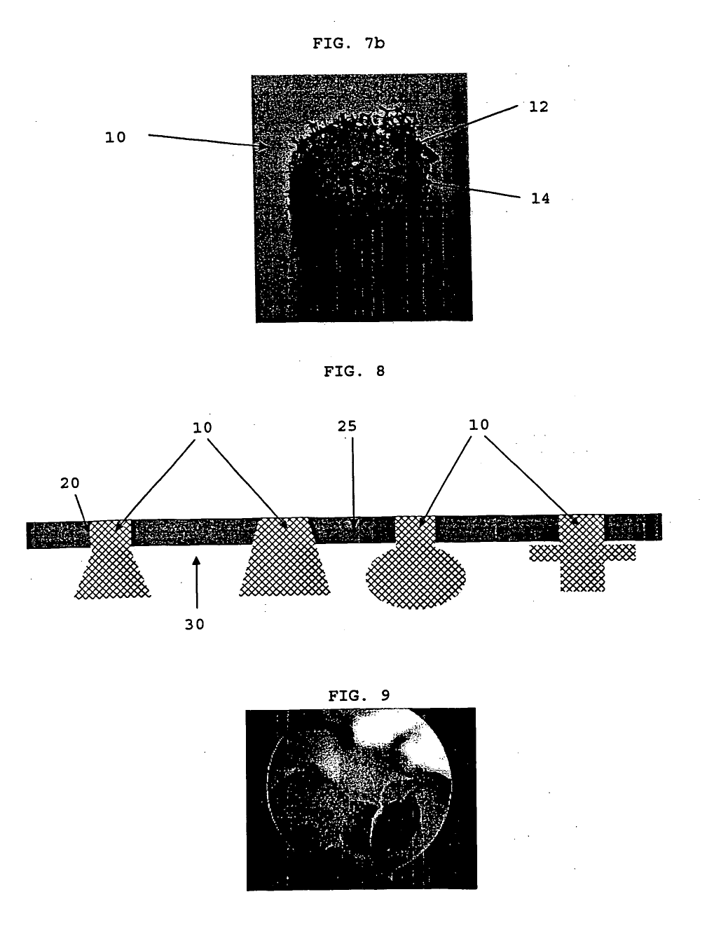

As illustrated in FIGS. 7a-b, in another embodiment, the first material 12 may be in the shape of a flat sheet and the second material 14 may be in the shape of a cylinder. The first material 12 may be wrapped around the second material 14 to form one example of a multiple piece implant 10 in which first material 12 may partially or substantially surround second material 14. Alternatively, first material 12 may be formed as a hollow cylinder, and second material 14 may be placed within the hollow interior and may be substantially surrounded.

Multiple piece implant 10 may be suitable for larger tissue defects, as well as odd-shaped defects, since the multiple pieces of the implant 10 can conform to the space of the defect better than a single piece implant. For example, the first material 12 may only wrap around a portion of second material 14, to partially surround second material 14, or first material 12 may amass towards one side of second material 14, to adapt to the space of the tissue defect.

Either of the first or second materials 12 or 14 may be composed of synthetic, natural, or recombinant material, or any combination thereof. The natural material may be of human, animal, and/or plant origin such as, for example, silk, collagen or hyaluronan-based material or the like. The synthetic material may be, for example, silk or a resorbable polymer, or a co-polymer, from the family of, for example, polycaprolactone, polyurethane, polyester, polyethylene, or the like, or a hyaluronan-based material. One naturally derived material which may be useful in the invention is DBM. The recombinant material may be collagen or silk.

In certain embodiments, the implant 10 is made of DBM, and the DBM may be processed to allow for variations in degree of demineralization throughout the implant 10, and even throughout at least one of the first or second materials 12 and 14. This may affect the compressible/expandable nature of the implant, so that its compressible nature may vary with location in the implant. This may be particularly advantageous in reconstructive procedures where structural rigidity of an implant is imperative.

For example, a devitalized cartilage matrix may be produced using a process similar to that used to create Osteosponge™. The starting material could be either cartilage only or could be an osteochondral core. Any source of cartilage cells could be used. Either could be processed to achieve a material that is expandable and/or compressible and appropriate for cartilage repair.

Besides the porous material for cartilage growth, the implant may, in one embodiment, include other portions, for example, a bone portion. The implant may consist, for example, of a cartilage portion extending into the bone portion of the defect. The implant may also consist of a bone portion extending into the cartilage portion of the defect. Alternatively, the implant may consist of two separate pieces, such as a first and second material, used in the same defect; a cartilage-appropriate portion and a separate bone-appropriate portion. The two portions may also be separated by a membrane to prevent fluid migration or may be used as delivery of biological factors.

In the example of the first material 12 and second material 14 both being made of DBM, the amount of demineralization of each material may be the same or may be different. For example, both the first material 12 and second material 14 may have the same degree of demineralization. Thus, both materials may have the same strength, porosity, compressibility and expandability specifications. Also, it can be expected that tissue ingrowth would occur at a constant rate throughout the entire implant 10.

However, if one material has a different degree of demineralization than the other material, strength, porosity, compressibility, expandability and tissue ingrowth rates of the two materials may differ. For example, porosity and tissue ingrowth may be higher in the first material 12 than in the second material 14, but the second material 14 would likely have greater strength due to higher demineralization in the second material 14 than in the first material 12.

Similarly, within at least one of the first material 12 or the second material 14, the degree of demineralization may change, creating a demineralization gradient through at least a portion of the first material 12 or the second material 14. For example, as to the first material 12, the degree of demineralization may form a demineralization gradient throughout the volume of material. In one embodiment, the gradient may occur from the bottom of the material 12 to the top. In another embodiment, the gradient may occur from the interior of the material 12 to the external surface. In a further embodiment, the gradient may differ from one side of the sheet to the other side of the sheet. The gradient, in one embodiment, may change in an axial or radial direction. A gradient such as these described would allow a single piece of implant 10, such as first material 12, to have both higher strength properties in one portion of first material 12 and higher porosity, tissue ingrowth, compressibility and expandability in another portion of first material 12.

Moreover, the multiple piece implant 10 not only provides greater flexibility to the surgeon since it conforms to the size and shape of the tissue defect, it may also be less expensive and more simple to manufacture than a single, large implant would be. Also, a single large implant would not be as flexible in its use as a multiple piece implant.

FIG. 8 illustrates some examples of the shapes of an implant. In FIG. 8, the exemplary implants shown are multiple piece implants 10 in the shapes of a cone, trapezoid, teardrop and a T-shape located with tissue defect site 20 (for FIGS. 8-10, each implant 10 will be located within a defect site 20, which is not designated in each figure or at every location for the sake of clarity). Of course, the actual shape of the implant 10 may be any of those shown, any variations of the ones shown, or any other shape which may be required. For example, the base of the T-shape may be wider or narrower than the central stem, or the T-shape may be reversed such that the base is located in the soft tissue or cartilage 25. The shapes illustrated in FIG. 8 may provide for increased fixation into a tissue, for example hard tissue 30. The increased fixation will ensure, for example, constant and firm interaction of the implant 10 with the cartilage or soft tissue 25 and the hard tissue 30.

Likewise, the above-referenced shapes may be multiple piece implants 10, and the implant 10 may be divided into multiple pieces in any way required. For example, in the T-shape, the arms of the “T” may be an annular disk which is combined with a separate rod forming the central stem. Also, the base of the “T” may be a single piece which is combined with a second piece making up the top stem portion. As mentioned above, the physical properties of each piece may be adjusted depending on the application such that the press-fit characteristics and the size of each piece, making up implant 10, may be specified.

While it has been described that the implant 10 should be adjusted to substantially fill the defect space, the aforementioned compressibility and expandability of the implant 10 may be utilized such that implants 10 of an initial shape different from the shape of the defect may be used to substantially fill the defect site. As one example, a hexagonal implant 10 may be used to substantially fill a cylindrical defect by compressing a hexagonal implant 10, which is initially larger than the defect site, to a size that is slightly smaller than the defect site. Once implanted, the implant 10 may be allowed to expand to substantially fill the defect site.

Additionally, more than one implant may be placed within a single defect site. For example, if multiple implants are placed within a single defect site, all of which are made of DBM, the implants may conform to each other to create intimate contact between each implant and to the surrounding tissue. This intimate contact may be generally continuous throughout the volume of the defect site and may further substantially fill the defect site, thus providing contact healing throughout the defect site along with a scaffold throughout which tissue may be regenerated. The multiple implants may be similar in shape to each other, or may be differently shaped from one another. FIG. 9 illustrates one example of multiple implants placed in a single defect site.

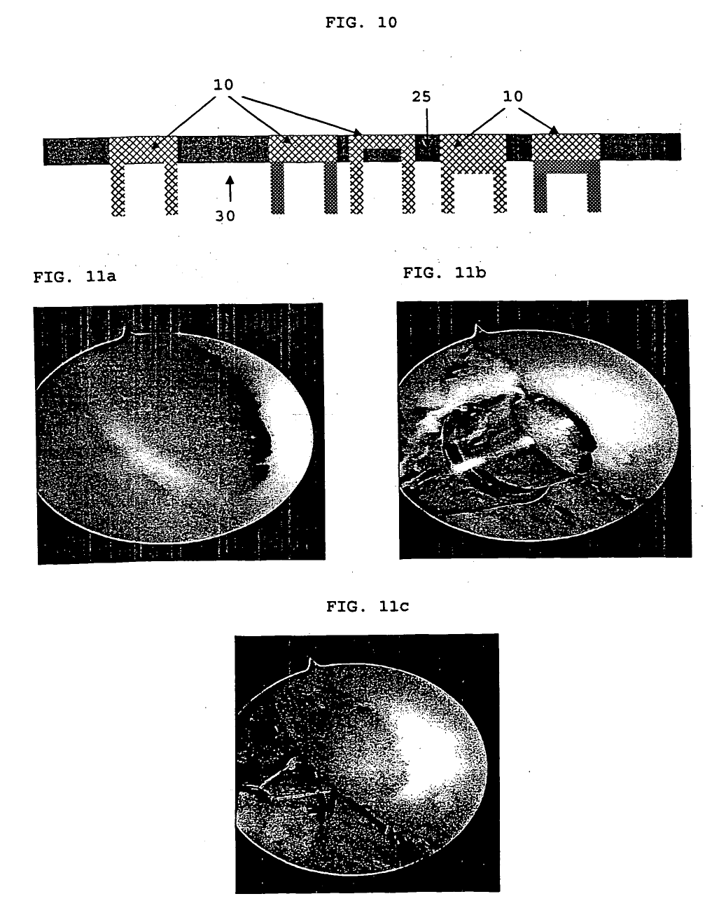

FIG. 10 illustrates yet another embodiment of implant 10. Implant 10, in this embodiment, is in the shape of a “tooth cap” which may include a horizontal portion and at least one “leg” extending in the vertical direction into hard tissue 30. This design may be made from a single piece or may be more than one piece. The horizontal portion may, if made of DBM, be substantially demineralized to have compressibility and porosity, while the legs may remain mineralized to provide strength in fixation with hard tissue 30. However, the legs may also be demineralized to provide additional compressibility and porosity.

The implant may, in some embodiments, be implanted into a defect site once the defect has been identified. The defect may first be cleaned, debrided and prepared. Tools may be used to form the defect site into a cylindrical shape, or alternatively into another shape such as an oval, square, rectangle, or another odd shape. In the case where a multiple piece implant, or multiple implants, is used, one piece may be added at a time into the defect. Once the first piece is implanted, it may be compressed, for example, radially, to make room for the implantation of a second piece. Once the second piece is within the defect, the first piece may be released, thus allowing the first and second pieces to come into intimate contact with one another and with the surrounding tissue. This method may be repeated as necessary until the entire defect is substantially filled. Alternatively, if a strip piece is used, it may be rolled up and compressed during insertion into the defect, and once within the defect, it may be released to intimately contact the surrounding tissue such that it substantially fills the defect and conforms to the shape and size of the defect.

Integration with the surrounding cartilage tissue 25 may not be commonly achieved when a typical, known “press-fit” plug is used. A tighter press-fit can be achieved by the expansion of the first material 12 of the invention inside the defect 20, and will enhance integration and improve the performance of the cartilage implant.

The second material 14, in one embodiment a graft, of the invention achieves better apposition with the surrounding cartilage tissue and decreases, or eliminates, micromotion. These results would be expected to yield improved healing of the cartilage defect and increased longevity. In addition, the implant 10 will provide a scaffold, which may be the first material 12, with improved fixation due to its ability to be compressed and expand inside the defect.

The material should be porous enough to allow cell growth. Each pore may be the same size, or the pores may be of varying sizes, so long as some of the pores are large enough to allow cell growth into the material. Additionally, the pores may vary or change in size on compression and/or expansion of the material. In certain embodiments, the material has pores with a diameter of at least about 10 microns, at least about 20 microns, at least about 30 microns, at least about 40 microns or at least about 50 microns. Larger size pores are also within the scope of the invention, for example at least about 75-1000 microns.

The material used in the invention is expandable and/or compressible by a significant amount. By “expandable by a significant amount” it is meant that the materials expand by at least about 5 or 10% of their original size. By “compressible by a significant amount” it is meant that the materials compress by at least about 5 or 10% of their original size.

In another embodiment, the first and second materials of the implant 10 may each expand by at least about 5 or 10% to at least about 300% of its original size. For example, the materials may expand by at least about 5%, 10%, 20%, 25%, 30%, 50%, 75%, 100%, 150%, 200%, 250%, or 300% of their original size. Likewise, each of the first and second materials may compress by at least about 10% to at least about 99% of its original size. For example, the materials may compress by at least about 5%, 10%, 20%, 25%, 30%, 50%, 75%, or 99% of their original size.

Either of the first or second materials may be bioresorbable, or non-resorbable. While non-resorbable implants may necessitate the need for an additional operative procedure, clinician control over the duration of time the implant remains intact could allow for increased integration of the implant into the defect site. The implant could be constructed to remain implanted for an indefinite period of time without negatively interfering in any biological processes or causing the patient pain.

The implant may be seeded with one or more types of cells prior to, at the time of, or after implantation. “Seeding” the implant with cells refers to the process of inserting, or placing, one or more types of cells into, or onto, at least a portion of the implant. The cells can be placed in or on the porous material of the implant, and can be placed on only one piece of the implant, a portion of one piece of the implant, or on the entire implant or any combination thereof. Likewise, different types of cells can be placed into different areas of the implant depending on the desire of the surgeon.

Suitable cells for seeding the implant include any kind of cartilage producing cells, or any kind of cells which may have a therapeutic affect, either in the implant or by migration out of the implant. Suitable cells include, but are not limited to embryonic stem cells, stem cells, bone marrow cells, mesenchymal cells, progenitor cells, chondroblasts, chondrocytes, osteoblasts, or combinations of these cells.

Any cells added to the implant can be retrieved from various sources, including the patient to be treated, other patients of the same species, pools of cells from other patients or animals, individual animals and commercially available cell lines. Cells may be unaltered and seeded onto implants immediately after removal from the source or remain in culture until being added to the implant. The cells may be allogenic, autogenic, or xenogenic to the patient to be treated. Combinations of cells may be used.

The implant may be used as an ex vivo matrix for cell growth and/or may be implanted in situ into a cartilage defect as an in vivo matrix for cell growth. The invention also comprises an implant produced by culturing with cells.

The implant may be cultured with appropriate cells ex vivo until the appropriate tissue forms and is then implanted, cultured with appropriate cells ex vivo and implanted before full tissue formation, or implanted without any culturing step at all.

One or more biological agents may be added to the implant, a piece of the implant, a portion of a piece of the implant or a portion of the implant. Likewise, different biological agents may be placed in various portions of the implant or may be placed simultaneously in various portions of the implant. By “biological agent” it is meant any agent that has, or produces, biological, physiological and/or pharmaceutical activity upon administration to a living organism. These biological agents may be added to the implant at any time, for example, before, during or after implantation.

The implant can have varying degrees of biological agent content. The presence of biological agents can be controlled such that growth factor content is maximal or negligible. Biological agent content may vary with depth or location.

Suitable biological agents include, but are not limited to, growth factors, cytokines, antibiotics, antimicrobials, biomolecules, drugs, strontium salts, fluoride salts, calcium salts, sodium salts, bone morphogenetic factors, chemotherapeutic agents, angiogenic factors, anti-inflammatory compounds, such as for example IL-1Ra or TNF-alpha, osteoconductive agents, chondroconductive agents, inductive agents, bisphosphonates, painkillers, proteins, peptides, or combinations thereof. Other biological agents may include cells such as for example allogenic cells, autologous cells, progenitor cells, stem cells, bone marrow stromal cells, mesenchymal cells, fibroblasts, chondrocytes, tenocytes, synovicytes, or the like. Further biological agents may include platelet-rich-plasma (PRP), platelet concentrate, bone marrow concentrate, plasma concentrate, blood, bone marrow, synovial fluid, hyaluronan and hyaluronic acid.

Growth factors that can be added to the implant include platelet derived growth factor (PDGF), transforming growth factor beta (TGFβ), insulin-related growth factor-I (IGF-I), insulin-related growth factor II(IGF-II), beta-2-microglobulin, bone morphogenetic proteins (BMPs), such as BMP-2, 4, or 7, fibroblast growth factor (FGF), hepatocyte growth factor (HGF), cartilage derived morphogenetic protein (CD-MP), growth differentiation factors (GDFs), or combinations of growth factors.

Chondroinductive agents include prostaglandin E2, thyroid hormone, dihydroxy vitamin D, ascorbic acid, dexamethasone, staurosporine, dibutyrl cAMP, concavalin A, vanadate, FK506, or combinations of different chondroinductive agents. Antibiotics include tetracycline hydrochloride, vancomycin, cephalosporins, and aminoglycocides such as tobramycin, gentamicin, and combinations thereof. Pain killers include lidocaine hydrochloride, bipivacaine hydrochloride, ketorolac tromethamine and other non-steroidal anti-inflammatory drugs.

The biological agent added to the implant may also be a protein or combinations of proteins. For example, proteins of demineralized bone, bone protein (BP), bone morphogenetic protein (BMP), BMP5, osteonectin, osteocalcin, osteogenin, or combinations of these proteins can be added to the implant.

Other suitable biological agents include cis-platinum, ifosfamide, methotrexate, doxorubicin hydrochloride, or combinations thereof.

Other materials such as gels, putties, cements or the like may also be added to the implant. Such materials, for example, may assist in securing the implant in place or to create separations between different pieces of the implant.

The above materials, biologics and cells may also be placed in between the multiple implant pieces which may enhance integration between the multiple pieces and the surrounding tissue.

The implant can be implanted dry or hydrated with liquids before, during, or after implantation. Examples of liquids include, but are not limited to water, saline, and bodily fluids (such as blood, bone marrow or synovial fluid). All or only part of the implant (for example, the porous material or part thereof) may be hydrated. The hydration may be done by any method, including dipping, sprinkling, full or partial submersion, running under a faucet, centrifugation through the scaffold, pressure, vacuum or negative pressure. The implant may be exposed to the liquid for an instant or up to several hours or several weeks, and can be stored in a liquid indefinitely until implantation.

The method of the invention can be used to treat any cartilage defect, whether it is in elastic cartilage, fibrocartilage, or hyaline cartilage. For example, the method could be used for cartilage repair in joints, such as a knee, ankle, hip, shoulder, elbow, temporomandibular, sternoclavicular, zygapophyseal, and wrist; or any other place where cartilage is found, such as the ear, nose, ribs, spinal column, pelvis, epiglottis, larynx, and windpipe. The implant may also be used in rhinoplasty procedures, including but not limited to reconstruction via a dorsal septal graft. The implant may be used to repair cartilage during a microtia-atresia surgical correction or in other types of auricular reconstructive procedures, such as those secondary to trauma or cancer. The implant may also be used to repair fibrocartilage found in, for example, the meniscus or labrum.

The implant of the invention can be used to repair cartilage in any patient in need thereof. By “patient” is meant any organism which has cartilage, including, but not limited to humans, monkeys, horses, goats, dogs, cats, and rodents.

One implant may be used alone to fill the defect, or multiple implants may be combined to fill one defect (similar to the mosaicplasty technique). In addition, the implant may be used to compliment other tissue repair procedures, including autograft, allograft, or mosaicplasty procedures. The implant of the invention may be implanted at the same time, before, or after other tissue repair procedures. The implant may also, in some embodiments, be multi-layered, such that, for example, the implant may have a cartilage layer and a bone layer, or the like.

The expandable/compressible material may be used to fill small gaps left during the other procedures. The implant can be used to fill either the donor or the recipient sites in mosaicplasty-like procedures, and can be used either alone or in combination with other materials, including allografts, autografts, other biomaterials or other grafts. For example, the first material 12 may be DBM which is porous, compressible and expandable, while the second material 14 may be an allograft or autograft. The first material 12 may help in integrating the graft, second material 14, with the surrounding tissues.

As discussed above, the implant may be produced in various shapes and sizes. The implant may be produced in a geometric shape, such as a flat sheet, square, rectangle, cylinder, pentagon, hexagon, T-shape, cone, tear-drop, tooth cap, or circle. The implant may also be produced to match the shape of all or part of an anatomical feature, such as an ear, nose, joint, knee, ankle, hip, shoulder, elbow, temporomandibular, sternoclavicular, zygapophyseal, wrist, rib, spinal column, pelvis, epiglottis, larynx, or windpipe.

A surgeon may alter the size of the implant material prior to implantation by means of scissors or some other instrument or device used for cutting. This gives the clinician the operative flexibility to customize the fit of the invention without detriment to the patient or the implant itself.

Prior to, after, or in the absence of compression, the implant can be shaped by the clinician to match any anatomical intricacies of the surgical implantation site. The implant can then be implanted, either dry or hydrated, via a procedure such as “press fit.” The implant can be compressed prior to implantation, or can be implanted without compression. The implant material may expand to substantially fill the defect after implantation.

An undersized void can be created in the tissue and possibly the adjacent bone where a defect is identified. For articulating joints, for example, the surgeon may create a defined defect in the articulating joint where fibrillation or a cartilage defect was identified. The defect may be chondral or osteochondral.

The implant, which can be oversized compared to the defect, may be compressed and implanted into the defect, either dry or hydrated. The implant may be compressed by any method, including by hand, by squeezing through a conical tube of a desired size, or via surgical instrument.

The implant may fill any void space by expanding to substantially fill the total volume of the defect. The constraint created by the undersized defect creates an increased press-fit with the surrounding tissue, enhanced integration and the elimination of micromotion. The implant may also be implanted without a press-fit or interference fit but will expand after implantation due to hydration with body fluids.

The implant may be merely press fit into the defect area or an anchor can be used to affix the implant to the defect. Anchors include plates, nails, screws, pins, tacks, adhesives, organic glues (such as fibrin glue), clotting materials or any other material known to be suitable for affixing soft tissue, cartilage, or bone grafts. More than one type of anchor may be used to affix the implant to the tissue defect site. Anchors such as these may be particularly useful for implantation of the implant into the meniscus to ensure a strong, tight fit to the underlying hard tissue adjacent the meniscus.

Because the implant can be compressible in all dimensions, it can be compressed to fit into small articulating joints, such as the hip. Thus, the ability to be compressed in three dimensions allows an implant to be used in the repair of tissue defects of the hip or other articulating joints or during arthroscopic surgeries.

Another embodiment of the invention is a variation of the press-fit technique. One challenge of certain procedures, particularly in the area of oral surgery is primary closure of the wound site post-osseous implant. This occurs when an osseous defect receives an implant intended to serve as a matrix for osseous regeneration. The surgeon faces the challenge of suturing the epithelial layer over the implant. The implant can be compressed and encapsulated in a bio-resorbable or non-resorbable capsule. The capsule can be made in a varying array of shapes and sizes. The capsule can be slightly smaller than the defect, or can be compressed to a size slightly smaller than the defect.

The capsule can be implanted into the defect and the surgeon sutures the epithelial tissue over the capsule inside of the defect creating a snug fit. The fit of the capsule should be tight enough to remain in place for suturing, but not occupy so much space as to make primary closure a challenge.

After closure, the blood and fluids in the defect can initiate bioresorbtion of the capsule allowing the material to expand to its full size within the defect. The fit of the material becomes tight with the borders of the defect, minimizing any micromotion within the defect.

The surgeon selects the size of the capsule and hydrated material based on the anatomical defect. Multiple capsules could be used if necessitated by the anatomical defect.

Instrumentation or imaging techniques to measure and match the cartilage defect and/or surgical instruments used in conjunction with graft implantation may be packaged with the graft as a kit.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention.

The entire disclosure of all references discussed herein is hereby incorporated by reference herein.

### EXAMPLE 1

Osteosponge™ (Bacterin) was used as the graft material in all examples.

In this example, an in vitro study was performed to quantify the expansion of a demineralized bone matrix sponge when hydrated with commercially available 1x phosphate buffered saline (PBS).

Hydration was conducted by manually compressing and submerging the sponge in PBS, until pliable. In an effort to reproduce surgical conditions, the sponge was hydrated at room temperature.

The diameter, thickness and volume of the sponge were measured 3 times. Measurements were taken when the sponge was dry, immediately after being hydrated for 1 hour, and immediately after being hydrated for 2 hours.

The percent change in the diameter, thickness and volume were calculated by comparing both the 1 hour measurements and 2 hour measurements to the dry measurements. The sponge expanded ˜15% in diameter, ˜11% in thickness and ˜45% in volume. The measurements taken at 1 hour and 2 hours were statistically equivalent. See Table 1.

TABLE 1

| | Diameter (mm) | Thickness (mm) | Volume (mm³) |
|---|---|---|---|
| dry | 5.01 ± 0.12 | 8.18 ± 0.30 | 161.14 ± 6.27 |
| hydrated | 5.77 ± 0.13 | 9.11 ± 0.59 | 238.48 ± 24.46 |
| 1 hour | | | |
| hydrated | 5.74 ± 0.13 | 9.04 ± 0.33 | 233.95 ± 16.23 |
| 2 hours | | | |
| % change | 14.57% ± 3.71% | 10.59% ± 3.68% | 45.37% ± 12.07% |

### EXAMPLE 2

In vitro studies were also conducted to demonstrate the ability of demineralized bone matrix (DBM) sponges to support chondrogenesis. The sponges were divided into two groups: sponges containing cells and sponges without cells.

Chondrocytes were harvested from the rear joints of goats under the age of 3 months old. The articular cartilage was harvested within 24 hours of death. Articular cartilage was collected from the patellar groove, femoral condyle, and patella.

Throughout harvesting, the tissue was bathed in PBS containing gentamicin (25 ug/mL). Cartilage tissue was digested using 0.2% collagenase (Worthington collagenase type 22, 2 mg collagenase per mL culture medium) for approximately 18 hours at 37° C. while shaking in an orbital shaker. The resulting cells were pelleted by centrifugation at 200 g for 10-15 minutes and strained through a 70 um cell strainer to separate the cells from cell debris and tissue fragments.

Following harvesting, the chondrocytes were seeded onto the sponges in 1 mL of cell culture medium (DMEM with 25 ug/mL gentamycin and 10% fetal bovine serum), at 37° C. in 24-well plates. The sponges were placed into the plates and the cell solution was placed on top of the sponges at a cell density of 30 million cells/cm³. The plates were shaken at 200 rpm for 18 hours.

After 18-24 hours of seeding, five cell-laden sponges from each experimental group were stained with MTT to assess cell distribution. Other cell-laden sponges were cultured in 6 well plates with 10 mL of culture medium comprising 10% FBS, 25 ug/ml of gentamicin and 50 ug/mL ascorbic acid for up to 6 weeks. The constructs were refed two to three times a week, and full refeeds were used (where all of the media is removed).

Sponges were analyzed at 3 weeks and 6 weeks for biochemical content, matrix uniformity and biomechanical properties.

DNA and glycosaminoglycan (GAG) content were assessed based on Hoechst 33258 and DMMB assays. GAG is a major constituent of the extracellular matrix of articular cartilage and indicates cartilage formation. See Table 2.

TABLE 2

| | % GAG (wet weight) |
|---|---|
| week 1 (N = 7) | 1.55 ± 0.53 |
| week 3 (N = 7) | 4.54 ± 2.04 |
| week 6 (N = 4) | 3.19 ± 0.50 |

The results prove a significant increase in GAG content in the cell-laden grafts thus indicating the presence of cartilage formation in the grafts containing chondrocytes.

Cross sections of grafts, both with and without chondrocytes, were stained with Safranin-O after six weeks in culture. Safranin-O is a red dye stain used to stain cellular nuclei in histological applications. Histological analysis of the samples revealed a cartilage like uniformity in the sponges containing chondrocytes, further supporting chondrogenesis in the cell-laden grafts.

### EXAMPLE 3

In the first in vivo study, the grafts were successfully implanted into defects created in the lateral and femoral condyle and trochlear grooves of goats. The femoral condyle was chosen because of its heavy weight bearing characteristics while the lateral groove was chosen because it is a lesser weight bearing site.

Tubular chisels were used to create and remove chondral and osteochondral cores measuring 4.5 mm in diameter. The remaining defects served as the implantation sites for grafts.

One graft consisting of DBM was hydrated with saline and implanted into each defect. Some grafts were combined with approximately 100-300 ul of fibrin glue according to manufacturer's instructions. Success was determined based on the ease of implantation, and whether the implanted grafts remained in the defect for the duration of the study.

### EXAMPLE 4

A second in vivo study examined the fixation of the grafts within an osteochondral defect after implantation.

The graft was initially hydrated with PBS. The graft was then compressed from a hydrated diameter of ˜6 mm in diameter into focal osteochondral defects of ˜4.5 mm. The grafts and defects were both ˜8 mm in depth.

Using the press-fit technique, the grafts were implanted into the lateral trochlear grooves and the medial femoral condyles of goats.

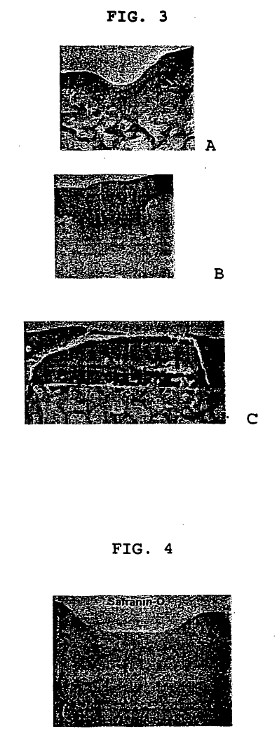

After 3 weeks, the animals were sacrificed, and the joints were histologically analyzed for the presence of the sponge. Safranin-O staining of cross sections of the joints containing sponges revealed remnants of the sponge still present in the sites of implantation. See FIG. 4.

### EXAMPLE 5

A third in vivo study examined the repair of focal osteochondral defects post-implantation. Results were examined after three months of implantation.

Two groups were studied in the current example and each group contained eight replicates. Each replicate was a goat femur containing two defects in the medial femoral condyle and two defects in the lateral trochlear groove.

For Group 1, osteochondral defects that received DBM grafts were compared to analogous defects that received autografts. For Group 2, osteochondral defects that received DBM grafts were compared to analogous defects that received microfracture.

Four defects were created using tubular chisels to create and remove osteochondral cores 4.5 mm wide and 8 mm deep. Two defects were made in the medial femoral condyle and two defects were made in the lateral trochlear groove of each replicate. The osteochondral grafts harvested from the first site at the condyle and the first site at the trochlear groove were disposed of. For Group 1, the grafts harvested from the second sites at the condyle and trochlear groove were implanted into the first defects at their respective locations. For Group 2, the full-thickness defect (articular and calcified cartilage removed) was created with a diameter of 4.5 mm. The defect was created using a tubular chisel, #15 scalpel blade and a currette. An awl was to create small holes in the subchondral bone, simulating microfracture in the goat. Perforations were made uniformly within the defect sites at an approximate depth of 3 mm.

The grafts, having initial hydrated diameters of ˜6.5 mm and widths of ˜8.5 mm, were compressed and implanted into the focal osteochondral defects employing the press-fit technique.

Post-implantation, the sponges protruded 0.5 mm proud to the adjacent cartilage. This technique is thought to aid in chondrogenesis.

After 3 months, the animals were sacrificed, and the joints were histologically analyzed for the presence of the sponge. Safranin-O staining of cross sections of the sites containing sponges revealed remnants of the sponge present in the implantation sites.

The presence of remnants of the sponges 3 months post-surgery proves the effectiveness of the technique in creating a sufficient fit between a sponge and the associated osseous or osteochondral defect. In comparison to the autograft-filled defects, the repair tissue in the DBM-filled defects shows a histological integration with the adjacent cartilage (FIGS. 3A and 3B), while the autografted sites demonstrated a gap between the osteochondral defect and the adjacent cartilage (FIG. 3C).

TABLE 3

| Group # | Objective | Timepoint | Defect #1 | Defect #2 |
|---|---|---|---|---|
| 1 | Effect of | 3 months | Scaffold in | Autograft |
| | scaffold in | | osteochondral | (positive |
| | osteochondral | | defect | control) |
| | defects | | | |
| 2 | Effect of | 3 months | Scaffold in | Microfracture |
| | scaffold in | | osteochondral | (clinical |
| | osteochondral | | defect | control) |
| | defects | | | |

### EXAMPLE 6

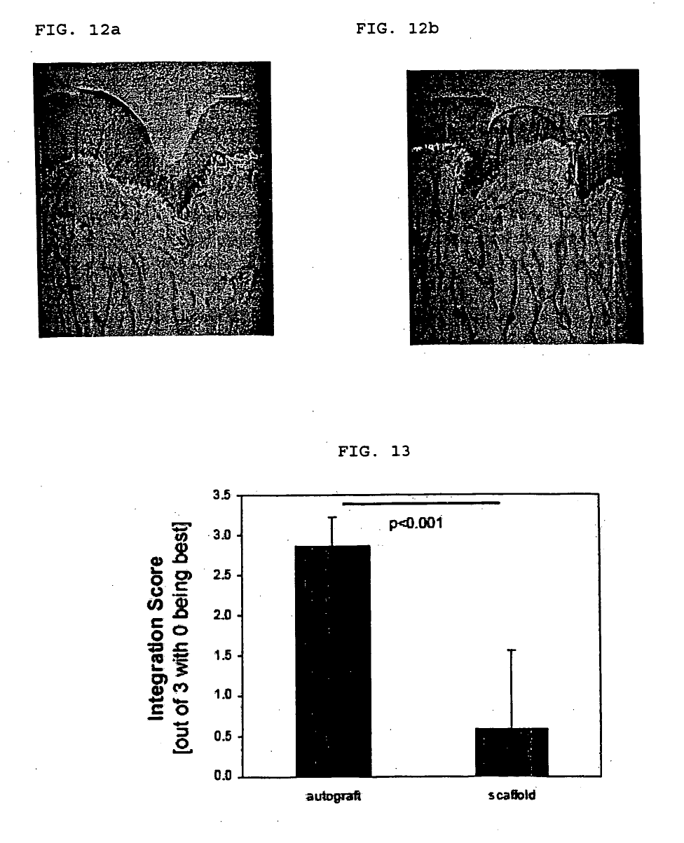

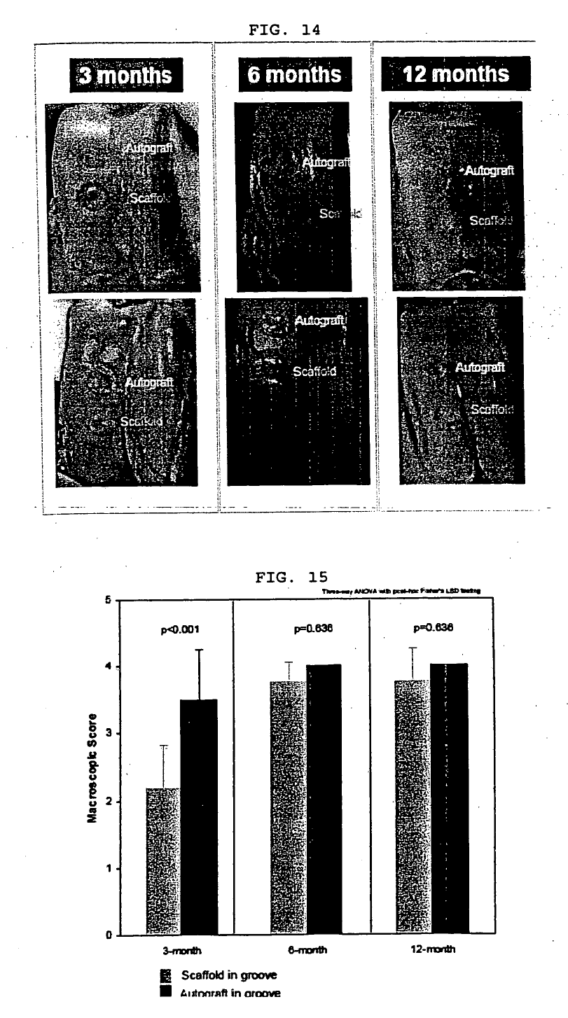

This Example is similar to Example 5, except the animals were sacrificed after 6 months and the joints were analyzed histologically and through the use of MRI, microCT and macroscopic methods for the presence of the sponge. FIGS. 11a-11c illustrate the implantation of the implant into the osteochondral defect. FIGS. 12a and 12b illustrate histological study of the implant (12a) as to an autograft (12b) implanted in the medial femoral condyle. The implant was shown to have much better integration than the autograft, illustrated in FIG. 13.

### EXAMPLE 7

This Example is similar to Examples 5 and 6, except the animals were sacrificed after 12 months and the joints were analyzed histologically and through the use of MRI, microCT and macroscopic methods for the presence of the sponge. FIG. 14 illustrates a macroscopic appearance of two examples of an autograft and an implant (labeled “scaffold”) at 3 months, 6 months, and 12 months in a trochlear groove defect. FIG. 15 illustrates the macroscopic score for both the implant and the autograft at 3 months, 6 months and 12 months.

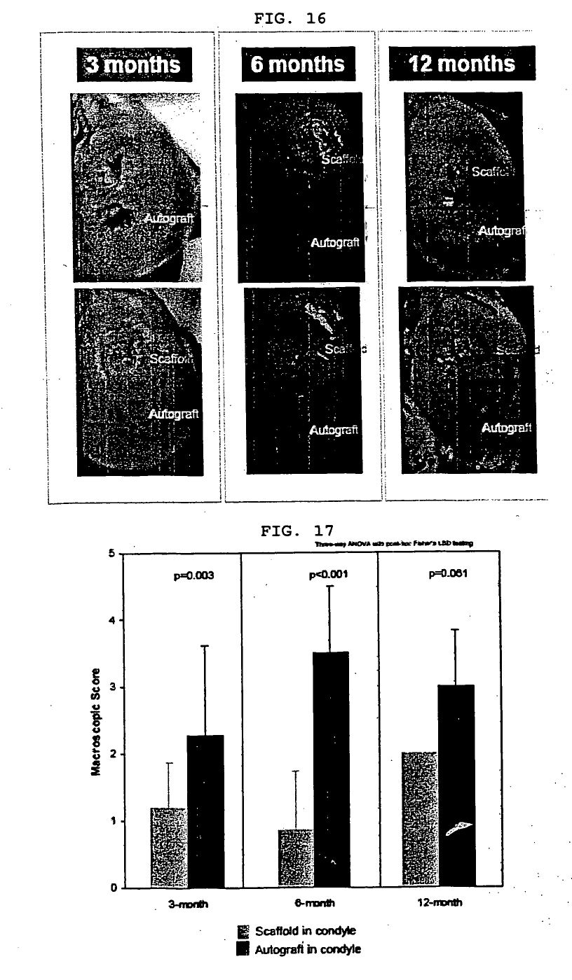

FIG. 16 illustrates a macroscopic appearance of two example of an autograft and an implant (labeled “scaffold”) at 3 months, 6 months, and 12 months in a condyle defect. FIG. 17 illustrates the macroscopic score for both the implant and the autograft at 3 months, 6 months and 12 months.

As in the previous examples, the integration of the implant into the defect is better than the integration of the autograft into the defect.

1. A method for the repair of a cartilage defect in a patient in need thereof, comprising implanting an implant into the cartilage defect, wherein said implant comprises a first porous material, wherein said first material is a scaffold that is expandable or compressible.

2. The method of claim 1, wherein said first material comprises a material selected from the group consisting of collagen and demineralized bone matrix.

3. The method of claim 1, wherein said implant further comprises a second material, wherein said second material is porous and is expandable or compressible.

4. The method of claim 3, wherein said second material comprises a material selected from the group consisting of collagen, demineralized bone matrix, allogenic tissue, xenogenic tissue, and synthetic material.

5. The method of claim 3, wherein at least one of said first material and said second material is expandable by at least 5% by volume.

6. The method of claim 3, wherein at least one of said first material and said second material is compressible by at least 5% by volume.

7. The method of claim 3, wherein at least a portion of one of said first material and second material comprises a flat sheet, square, rectangle, cylinder, pentagon, hexagon, geometric shape, T-shape, cone, tear-drop, tooth cap, spiral, snail-shape, centipede, or circular-shaped structure.

8. The method of claim 1, wherein said implant further comprises at least one biological agent.

9. The method of claim 1, wherein said implantation is performed before, after, or at the same time as a procedure selected from the group consisting of mosaicplasty, autograft, or allograft.

10. The method of claim 1, wherein said implant further comprises cells selected from the group consisting of embryonic stem cells, stem cells, bone marrow cells, mesenchymal cells, progenitor cells, chondroblasts, chondrocytes, osteoblasts, and combinations of these cells.

11. The method of claim 1, where said defect is located in a site selected from the group consisting of knee, ankle, hip, shoulder, elbow, temporomandibular, sternoclavicular, zygapophyseal, wrist, ear, nose, ribs, spinal column, pelvis, epiglottis, larynx, and windpipe.

12. The method of claim 1, wherein more than one implant is implanted into said cartilage defect.

13. The method of claim 1, wherein said implant is fixed to the defect with an anchor selected from the group consisting of glue, adhesive, a screw, a tack and a nail.

14. An implant for the repair of a cartilage defect in a patient in need thereof, the implant comprising a first porous material and a second material, wherein said first material is a scaffold that is expandable or compressible, and wherein said first material partially or substantially surrounds said second material.

15. The implant of claim 14, wherein said first material comprises a material selected from the group consisting of collagen and demineralized bone matrix.

16. The implant of claim 14, wherein said second material is porous and is expandable or compressible.

17. The implant of claim 16, wherein at least one of said first material and said second material is at least one of expandable or compressible by at least 5% by volume.

18. The implant of claim 14, wherein said second material comprises a material selected from the group consisting of collagen, demineralized bone matrix, allogenic tissue, xenogenic tissue, and synthetic material.

19. The implant of claim 18, wherein said second material comprises demineralized bone matrix, and said first material comprises demineralized bone matrix, and said first material has a degree of demineralization that is either the same as, greater than, or less than the degree of demineralization of said second material.

20. The implant of claim 14, wherein said demineralized bone matrix of said first material has a demineralization gradient that is selected from the group consisting of constant throughout the entire volume of said first material and variable throughout the entire volume of said first porous material.
